(19) United States
(12) Patent Application Publication
(10) Pub. No.: US 2023/0024842 A1
(43) Pub. Date: Jan. 26, 2023

(54) MUPS TABLET COMPRISING RIBOFLAVIN

(71) Applicant: DSM IP Assets B.V., HEERLEN (NL)

(72) Inventor: Elger FUNDA, Kaiseraugst (CH)

(21) Appl. No.: 17/779,702

(22) PCT Filed: Nov. 23, 2020
(86) PCT No.: PCT/EP2020/083017

§ 371 Date: May 25, 2022

(30) Foreign Application Priority Data
Nov. 27, 2019 (EP) 19211695.2

(51) Int. Cl. A61K 9/20; C07K 16/24; A61K 38/17; A61K 31/525

(52) U.S. Cl. CPC A61K 9/2081; C07K 16/241; A61K 38/1793; A61K 31/525; A61K 9/205; A61K 9/2068; A61K 9/2009; A61K 9/2027

(57) ABSTRACT

The present invention relates to a new formulation of specific nutritional ingredients (nutraceuticals) and/or pharmaceutical compounds.

The present invention relates to a new formulation of specific nutritional ingredients (nutraceuticals) and/or pharmaceutical compounds.

The present invention is related to a formulation (mainly for oral consumption), which comprises riboflavin (as a nutritional ingredient) and (usually) a pharmaceutical compound, which is used to treat (the symptoms of) IBD (such as TNF (Tumor Necrosis Factor) inhibitors).

When riboflavin (also known as vitamin B2) is used in a formulation, it will show an intensive yellow color, when it is dissolved in water or even when it comes in contact with water. This leads to an unpleasant coloration of the mouth and/or the tongue of a patient who is consuming such a formulation.

The goal of the present invention was to provide a formulation (mainly for oral consumption)

The formulation for the oral consumption (usually a tablet) could be coated to solve the problem of the unwanted coloration. However, in case the specific nutritional ingredients (nutraceuticals) and/or pharmaceutical compounds are to be delivered to the small or large intestine a regular coating of the tablet might not be the ideal solution, e.g. due to large variations of residence time in the stomach or dose dumping effect.

There is a need of formulations, which do not have the disadvantages are described above.

Therefore, it was found that the coloration issue of the riboflavin (in combination with the TNF inhibitors) can be solved by using a multiple-unit pellet system (MUPS), wherein the riboflavin particles are coated.

Riboflavin, also known as vitamin B2, is a micronutrient with a key role in maintaining health in humans and other mammals. It is the central component of the cofactors FAD and FMN, and is therefore required by all flavoproteins. As such, riboflavin is required for a wide variety of cellular processes. It plays a key role in energy metabolism, and for the metabolism of fats, ketone bodies, carbohydrates, and proteins. Riboflavin is found naturally in asparagus, popcorn, bananas, per-simmons, okra, chard, cottage cheese, milk, yogurt, meat, eggs, fish, and green beans. Other sources specify cheese, leafy green vegetables, liver, kidneys, legumes, tomatoes, yeast, mushrooms, and almonds. Recently it has been shown that Riboflavin has a beneficial effect on growth of Faecalibacterium prausnitzii, which is a marker species for a healthy gut.

TNF (Tumor Necrosis Factor) inhibitors (also known as tnf inhibitors-alfa inhibitors, tnf-alpha inhibitors) are a group of medicines that suppress the body's natural response to tumor necrosis factor (TNF), a protein produced by white blood cells that is involved in early inflammatory events.

TNF is involved in autoimmune and immune-mediated disorders such as rheumatoid arthritis, ankylosing spondylitis, inflammatory bowel disease, psoriasis, hidradenitis suppurativa and refractory asthma, so TNF inhibitors may be used in their treatment.

Inhibition of TNF effects can be achieved with a monoclonal antibody such as infliximab (Remicade), adalimumab (Humira), certolizumab pegol (Cimzia), and golimumab (Simponi), or with a circulating receptor fusion protein such as etanercept (Enbrel).

Thalidomide (Immunoprin) and its derivatives lenalidomide (Revlimid) and pomalidomide (Pomalyst, Imnovid) are also active against TNF.

While most clinically useful TNF inhibitors are monoclonal antibodies, some are simple molecules such as xanthine derivatives (e.g. pentoxifylline) and bupropion.

Several 5-HT2A agonist hallucinogens including (R)-DOI, TCB-2, LSD and LA-SS-Az are also inhibitors of TNF.

There is a range of commercially available TNF inhibitors on the market. The most important ones are the following ones (in brackets are the commercial drug forms):
Adalimumab (Humira®)
Certolizumab pegol (Cimzia®)
Etanercept (Enbrel®)
Golimumab (Simponi®)
Infliximab (Remicade®)

Multiple unit pellet systems (MUPS) are well known in the field of pharmaceutical applications. They are described in many patent applications as well as scientific papers. Also the production of MUPS is described and well known. MUPS as therapeutical dosage forms can either be used as such, e.g. as granulate in a sachet or they can be integrated in other dosage forms like tablets or capsules.

The present invention relates to a multiple unit pellet system (MU), which comprises at least two different particles
(a) pellets (pellets (a)) comprising 10-90 weight-% (wt-%), based on the total weight of the pellet (a), of riboflavin and
(b) particles (particles (b)) comprising at least one TNF inhibitor,
wherein the pellets (a) are coated.

The content of riboflavin in the multiple unit pellet system is usually always at least 1 wt %, based on the total weight of the multiple unit pellet system. Usually is between 1-50 wt-%, preferably between 2-40 wt-%, more preferably 4 to 35 wt-%.

The content of riboflavin in the multiple unit pellet system is usually always at least 1 wt %, based on the total weight of the multiple unit pellet system. Usually is between 1-50 wt-%, preferably between 2-40 wt-%, more preferably 4 to 35 wt-%.

Therefore, the present invention relates to a multiple unit pellet system (MU′), which is the multiple unit pellet system (MU), wherein the content of riboflavin in the multiple unit pellet system is at least 1 wt-%, based on the total weight of the multiple unit pellet system.

Therefore, the present invention relates to a multiple unit pellet system (MU″), which is the multiple unit pellet system (MU), wherein the content of riboflavin in the multiple unit pellet system is between 1-50 wt-%, based on the total weight of the multiple unit pellet system.

Therefore, the present invention relates to a multiple unit pellet system (MU′″), which is the multiple unit pellet system (MU), wherein the content of riboflavin in the multiple unit pellet system is between 2-40 wt-%, based on the total weight of the multiple unit pellet system.

Therefore, the present invention relates to a multiple unit pellet system (MU″″), which is the multiple unit pellet system (MU), wherein the content of riboflavin in the multiple unit pellet system is between 4-35 wt-%, based on the total weight of the multiple unit pellet system.

The overall content of the TNF inhibitor in the MUPS according to the present invention can vary and is should be present in an effective amount.

In the multiple unit pellet system according to the present invention, the weight ratio of the pellet (a) to pellet (b) can vary. Usually it the weight ratio goes from 5:1 to 1:5, preferably 4:1 to 1:4, more preferably 3:1 to 1:3, especially preferred from 2:1 to 1:2. Also preferred is a weight ratio of 1:1.

As stated above the overall content of riboflavin in the multiple unit pellet system according to the present invention is always at least 1 wt-%, based in the total weight of the multiple unit pellet system. Usually it is between 1-50 wt-%, preferably between 2-40 wt-%, more preferably 4 to 35 wt-%.

Therefore, the present invention relates to a multiple unit pellet system (MU1), which is the multiple unit pellet system (MU), (MU′), (MU″), (MU′″) or (MU″″), wherein the weight ratio of the pellet (a) to particle (b) is 5:1 to 1:5.

Therefore, the present invention relates to a multiple unit pellet system (MU1′), which is the multiple unit pellet system (MU), (MU′), (MU″), (MU′″) or (MU″″), wherein the weight ratio of the pellet (a) to particle (b) is 4:1 to 1:4.

Therefore, the present invention relates to a multiple unit pellet system (MU1″), which is the multiple unit pellet system (MU), (MU′), (MU″), (MU′″) or (MU″″), wherein the weight ratio of the pellet (a) to particle (b) is 3:1 to 1:3.

Therefore, the present invention relates to a multiple unit pellet system (MU1′″), which is the multiple unit pellet system (MU), (MU′), (MU″), (MU′″) or (MU″″), wherein the weight ratio of the pellet (a) to particle (b) is 2:1 to 1:2.

Therefore, the present invention relates to a multiple unit pellet system (MU1″″), which is the multiple unit pellet system (MU), (MU′), (MU″), (MU′″) or (MU″″), wherein the weight ratio of the pellet (a) to particle (b) is 1:1 to 1:1.

A preferred embodiment is a multiple unit pellet system, wherein the TNF inhibitor is chosen from the group consisting of adalimumab, certolizumab pegol, etanercept, golimumab and infliximab.

Therefore, the present invention relates to a multiple unit pellet system (MU2), which is the multiple unit pellet system (MU), (MU′), (MU″), (MU′″), (MU″″), (MU1), (MU1′), (MU1″), (MU1′″) or (MU1″″), wherein the TNF inhibitor is chosen from the group consisting of adalimumab, certolizumab pegol, etanercept, golimumab and infliximab.

Pellets (a), which comprise riboflavin comprise between 10-90 wt-% based on the total weight of the pellet (a) of riboflavin. Preferably 15-75 wt-%.

The pellet (a) can comprise any other commonly used ingredient to form a pellet. Such ingredients are known in the field of technology.

Therefore, the present invention relates to a multiple unit pellet system (MU3), which is multiple unit pellet system (MU), (MU′), (MU″), (MU′″), (MU″″), (MU1), (MU1′), (MU1″), (MU1′″), (MU1″″) or (MU2), wherein pellets (a), comprise between 15-75 wt-% based on the total weight of the pellet (a), of riboflavin.

A further embodiment of the present invention also relates to a multiple unit pellet system (MU4), which comprises at least two different particles
(a) pellets (pellets (a)) comprising riboflavin and
(b) particles (particles (b)) comprising at least one TNF inhibitor,
wherein the pellets (a) are coated,
   and which is free (does not comprise) any aminosalicylate or any pharmaceutically acceptable salt or any one prodrugs thereof.

Therefore, the present invention relates to a multiple unit pellet system (MU4′), which is multiple unit pellet system (MU4), wherein pellets (a), comprise between 10-90 wt-% based on the total weight of the pellet (a), of riboflavin.

Therefore, the present invention relates to a multiple unit pellet system (MU4″), which is multiple unit pellet system (MU4), wherein pellets (a), comprise between 15-75 wt-% based on the total weight of the pellet (a), of riboflavin.

In the multiple unit pellet system according to the present invention, the weight ratio of the pellet (a) to particle (b) can vary. Usually it the weight ratio goes from 5:1 to 1:5, preferably 4:1 to 1:4, more preferably 3:1 to 1:3, especially preferred from 2:1 to 1:2. Also preferred is a weight ratio of 1:1.

As stated above the overall content of riboflavin in the multiple unit pellet system according to the present invention is always at least 1 wt-%, based in the total weight of the multiple unit pellet system. Usually it is between 1-50 wt-%, preferably between 2-40 wt-%, more preferably 4 to 35 wt-%.

Therefore, the present invention relates to a multiple unit pellet system (MU5), which is the multiple unit pellet system (MU4), (MU4′) or (MU4″), wherein the weight ratio of the pellet (a) to particle (b) is 5:1 to 1:5.

Therefore, the present invention relates to a multiple unit pellet system (MU5′), which is the multiple unit pellet system (MU4), (MU4′) or (MU4″), wherein the weight ratio of the pellet (a) to particle (b) is 4:1 to 1:4.

Therefore, the present invention relates to a multiple unit pellet system (MU5″), which is the multiple unit pellet system (MU4), (MU4′) or (MU4″), wherein the weight ratio of the pellet (a) to particle (b) is 3:1 to 1:3.

Therefore, the present invention relates to a multiple unit pellet system (MU5′″), which is the multiple unit pellet system (MU4), (MU4′) or (MU4″), wherein the weight ratio of the pellet (a) to particle (b) is 2:1 to 1:2.

Therefore, the present invention relates to a multiple unit pellet system (MU5″″), which is the multiple unit pellet system (MU4), (MU4′) or (MU4″), wherein the weight ratio of the pellet (a) to particle (b) is 1:1 to 1:1.

Furthermore, the present invention also relates to a multiple unit pellet system (MU6), which comprises at least two different particles
(a) pellets (pellets (a)) comprising riboflavin and
(b) particles (particles (b)) consisting of at least one TNF inhibitor,
wherein the pellets (a) are coated.

Therefore, the present invention relates to a multiple unit pellet system (MU6′), which is multiple unit pellet system (MU6), wherein pellets (a), comprise between 10-90 wt-% based on the total weight of the pellet (a), of riboflavin.

Therefore, the present invention relates to a multiple unit pellet system (MU6″), which is multiple unit pellet system (MU6), wherein pellets (a), comprise between 15-75 wt-% based on the total weight of the pellet (a), of riboflavin.

Particles (b), can be any kind of particles (such as powders, granules, beadlets and pellets, as well as any mixture of them) which comprise the at least one TNF inhibitor can comprise between 0.5-95 wt-% based on the total weight of the particle (b), of at least one TNF inhibitor. Preferably 1-50 wt-%. (the content depends on the kind of the particle (powder do have a much higher content than a beadlet for example).

Therefore, the present invention relates to a multiple unit pellet system (MU7), which is multiple unit pellet system (MU), (MU′), (MU″), (MU′″), (MU″″), (MU1), (MU1′), (MU1″), (MU1′″), (MU1″″), (MU2), (MU3), (MU3′), (MU4), (MU4′), (MU4″), (MU5), (MU′5), (MU5″), (MU5′″), (MU5″″), (MU6), (MU6′) or (MU6″), wherein particles (b) comprise between 0.5-95 wt-% based on the total weight of the particle (b), of at least one TNF inhibitor.

Therefore, the present invention relates to a multiple unit pellet system (MU7′), which is multiple unit pellet system ((MU), (MU′), (MU″), (MU′″), (MU″″), (MU1), (MU1′), (MU1″), (MU1′″), (MU1″″), (MU2), (MU3), (MU3′), (MU4), (MU4′), (MU4″), (MU5), (MU′5), (MU5″), (MU5′″), (MU5″″), (MU6), (MU6′) or (MU6″), wherein particles (b) comprise between 1-50 wt-% based on the total weight of the particle (b), of at least one TNF inhibitor.

The overall dosage of the one TNF inhibitor, depends on the galenical form of the MUPS system and also on the daily dosage regime of the galenical form. This means that the content of the thiopurine (and/or at least a pharmaceutically acceptable salt and/or at least one prodrug thereof), can vary a lot.

Daily dose recommendations for the thiopurine (and/or at least a pharmaceutically acceptable salt and/or at least one prodrug thereof) vary also. Usually it is between 2.5 and 20 mg/kg body weight (this depends on the TNF inhibitor). More details can be found in S. O. Adegbola et al; Int. J. Mol. Sci 2018 (19), 2244. Therefore the overall amount of the at least one TNF inhibitor and/or at least a pharmaceutically acceptable salt and/or at least one prodrug thereof in the multiple unit pellet system according to the present invention can vary depending on the galenical form of the MUPS system and also on the (daily) dosage regime of the galenical form.

Suitable coating materials for the pellets are such, which release the riboflavin (and if coated also the at least one TNF inhibitor) in the small or large intestine.

Suitable coating material are polymers, which are derivatives of acrylic acid and cellulose. Various pH-dependent coating polymers include cellulose acetate phthalate (CAP) (Aquateric®), poly vinyl acetate phthalate (PVAP) (Coateric®), hydroxypropyl methyl cellulose phthalate (HPMCP), and methacrylic acid copolymers, commonly known as methacrylate copolymers or Eudragit.

Shellac and fats are also suitable coating materials.

Also suitable materials for the coating are for example alginate, chitosan, pectin, cyclodextrin as well as other gums. Preferred are alginate or pectin. This kind of coating might be crosslinked. The crosslinking can be done by commonly known crosslinking compounds. In case alginate is used that can be done by Mg and/or Ca ion (by the use of a salt). The crosslinker can be sprayed onto to pellet after having applied coating material or simultaneously. Or the coated pellets can be dipped into a solution comprising the crosslinker. Preferably the crosslinker is sprayed onto the particles after having applies the coating layer.

The coating layer is usually covering the pellet (more or less) completely.

Typically, the layer thickness of the coating layer is at least 10 μm. Preferably, thickness of the coating layer is at least 50-70 μm. Required layer thickness is determined by barrier properties of the coating material. To achieve such coating layer thickness, the amount of coating material is at least 10% (w/w) of the coated particle. Typically, the amount of coating material is at least 20% (w/w) of the coated particle.

The coating of pellet (a) is about 5-60 wt-%, based on the total weight of the pellet (a). In case particle (b) is coated then the coating is about 5-60 wt-%, based on the total weight of the particle (b).

It is clear, that MUPS tablets according to the present invention comprises the usual auxiliary ingredients, which are needed to form tablet.

Such auxiliary ingredients are for example, binders, fillers, lubricants, proteins, dyes, flavors, sweeteners, minerals, and antioxidants without being limited thereto.

Particularly suitable fillers according to the present invention encompass mono-, di- and tri-calcium phosphate, limestone (calcium carbonate), magnesium carbonate, silicate compounds (magnesium and aluminum silicate), magnesiumoxide, microcrystalline cellulose, proteins, silicon dioxide as well as mixtures thereof, such as more in particular microcrystalline wax, microcrystalline cellulose and limestone as well as mixtures thereof, most preferably microcrystalline cellulose.

Particularly suitable lubricants according to the present invention are water insoluble lubricants and encompass magnesium stearate, calcium stearate, zinc stearate or stearic acid such as more in particular magnesium stearate and/or calcium stearate.

If present, the total amount of the auxiliary ingredients can be up to 99 wt-%, based on the total weight of the MUPS tablet. Usually between 10-80 wt-%.

The MUPS tablets according to the present invention are compressed tablets, which depending on the process of production as well as the storage conditions, may comprise some water. Generally, the moisture content of the tablets according to the present invention is below 5 wt-%, based on the total weight of the MUPS tablet.

The MUPS tablet usually comprises up to 40 wt.-%, based on the total weight of the MUPS tablet, of pellets,

Preferably MUPS tablets according to the invention are preferably un-coated. If needed or wish the MUPS tablets could also be coated

More preferably MUPS tablets according to the invention are having a tablet weight of 100 to 1000 mg, preferably 300 to 900 mg.

The MUPS tablets according to the invention are produced by the following process
(i) the pellets (a) and the powder or particles (b) are produced, and
(ii) the pellets (a) and optionally the particles (b) are coated, and
(iii) the pellets and the particles are mixed, and
(iv) optionally other ingredients, which are useful or desired to form the tablet are added, and
(v) compressing the mixture obtained in step (iii) or step (iv) into a tablet.

The pharmaceutical compositions according to the invention are intended for oral use and can be used in the dosage form of an uncoated MUPS tablet or a film-coated MUPS tablet.

A further object of the invention are MUPS tablets obtainable by a process according to the invention.

The MUPS tablets can be of any size and shape, preferably the MUPS tablets can be of sizes from 21.0×10.0×9.0 to 11.0×5.0×3.0 mm, preferably from 21.0×10.0×9.0 to 14.0×6.0×4.0 mm, most preferred from 21.0×10.0×8.0 mm to 15.0×7.0×4.0 mm.

It also possible that the pellet (b) are coated. Usually the same kind of coating material as described and disclosed for the pellets (a) are used. It is not essential that the pellets (a) and the pellets (b) are the identical coating material when used in a MUPS tablet.

The same applies for pellets (a). It possible to use more than one coating material. This means that a certain amount of the pellets is coated with one coating material, whereas another amount of the pellets are coated with another coating material.

The following examples serve to illustrate specific embodiments of the invention claimed herein. All percentages are given in relation to the weight and all the temperatures are given in degree Celsius.

### EXAMPLES

### Example 1: Coating with Alginate/Shellac

80 g granulated Riboflavin was successively coated with 9% Na-alginate, 1% Ca-Chloride and 25% shellac using a WFP-mini fluid bed processor (DMR) in Wurster configuration. 89 g coated product with particle size between 250 and 1000 μm was obtained. Coating material was 35% of the particle mass, riboflavin content 50%.

20 mg of coated powder (containing 10 mg of Riboflavin) was dissolved in 1000 ml water for 15 min. Absorption of the solution was measured at 545 nm using a spectrophotometer (Genesys 20, thermo Scientific). Absorption of the solution was 0.065.

### Comparison Example 2: Solution of Uncoated Riboflavin

10 mg of riboflavin powder was dissolved in 1000 ml water for 15 min. Absorption of the solution was measured at 545 nm using a spectrophotometer (Genesys 20, thermo Scientific). Absorption of the solution was 0.386.

### Example 3: Coating with Eudragit FS30D

80 g granulated Riboflavin was coated with 60 g Eudragit FS30D and 10 g PlasACRYL T20 using a WFP-mini fluid bed processor (DMR) in Wurster configuration. 71 g coated product with particle size between 250 and 1000 μm was obtained. Coating material was ca. 20% of the particle mass, riboflavin content was 60%.

16.6 mg of coated powder (containing 10 mg of Riboflavin) was dissolved in 1000 ml water for 15 min. Absorption of the solution was measured at 545 nm using a spectrophotometer (Genesys 20, thermo Scientific). Absorption of the solution was 0.087.

1. A multiple unit pellet system, which comprises at least two different pellets
(a) pellets (pellets (a)) comprising and
(b) particles (particles (b)) comprising at least one TNF inhibitor,
wherein the pellets (a) are coated.

2. Multiple unit pellet system according to claim 1, wherein the TNF inhibitor is chosen from the group consisting of adalimumab, certolizumab pegol, etanercept, golimumab and infliximab.

3. Multiple unit pellet system according to claim 1, which is in the form of a tablet.

4. Multiple unit pellet system according to claim 1, which comprises at least one auxiliary ingredient.

5. Multiple unit pellet system according to claim 4, wherein the at least one auxiliary ingredient is chosen from the group consisting of binders, fillers, lubricants, proteins, dyes, flavours, sweeteners, minerals, and antioxidants without being limited thereto

6. Multiple unit pellet system according to claim 4, wherein the total amount of the auxiliary ingredients is up to 99 wt-%, based on the total weight of the MUPS tablet.

7. Multiple unit pellet system according to claim 1, wherein the coating material is chosen from the group consisting of cellulose acetate phthalate (CAP) (Aquateric®), poly vinyl acetate phthalate (PVAP) (Coateric®), hydroxypropyl methyl cellulose phthalate (HPMCP), and methacrylic acid copolymers, shellac, fats alginate, chitosan, pectin, cyclodextrin and other gums.

8. Process for the production of the multiple unit pellet system according to claim 1, comprising the following step:
(i) the pellets (a) and the particles (b) are produced, and
(ii) the pellets (a) and optionally the particles (b) are coated, and
(iii) the pellets and the particles are mixed, and
(iv) optionally other ingredients, which are useful or desired to form the tablet are added, and
(v) compressing the mixture obtained in step (iii) or step (iv) into a tablet.
